(19) United States
(12) Patent Application Publication
(10) Pub. No.: US 2011/0152814 A1
(43) Pub. Date: Jun. 23, 2011

(54) NON-WOVEN BIODEGRADABLE HYGIENE PRODUCT

(75) Inventor: Charishma Mohini Wickremesinghe Seneviratne, Como (AU)

(73) Assignee: SENEVENS INTERNATIONAL LTD., Como (AU)

(21) Appl. No.: 12/679,077

(22) PCT Filed: Sep. 19, 2007
(86) PCT No.: PCT/AU2007/001375

§ 371 Date: Nov. 19, 2010

(51) Int. Cl. A61L 15/28

(52) U.S. Cl. 604/375

(57) ABSTRACT

A non-woven biodegradable disposable personal product such as a diaper including an absorbent core formed of starch grains or flakes and hydrogel dispersed in viscose fluff. The personal product further includes an outer cover, a hydrophobic layer, a biodegradable film and a hydrophilic layer. The hydrophobic layer, the biodegradable film and the hydrophilic layer are all formed of polylactic acid (PLA).

### FIELD OF THE INVENTION

The present invention relates to a disposable non-woven substantially biodegradable personal product and an absorbent composition for use therewith. The invention has been devised particularly, but not solely, for use in absorbing body waste discharges, and typically includes single use products such as infant diapers, feminine hygiene products, incontinence products, and other like products.

### BACKGROUND OF THE INVENTION

Articles used to absorb waste body fluids such as urine, are used in large quantities worldwide. Typically these articles are in the form of disposable diapers, and other like products such as feminine hygiene products and incontinence products, which have become increasingly popular as a modern convenience and necessity.

Large quantities of disposable infant diapers are used each year and disposal of these products is a problem. Most of the commercially available disposable diapers consist largely of plastics based on polypropylene and polyethylene which do not break down in the environment, that is they resist biodegradation totally.

A further difficulty associated with prior art disposable diapers is that due to the large percentages of toxic chemicals in these products, rashes and skin allergies often arise in the user. Due to the requirements to maximise the absorbency of these products, the quantities of added toxic chemicals, such as polyacrylate polymers, have been increased, further compounding the associated problems.

A number of prior art diapers are uncomfortable to wear as they are made of plastic material, and are paper based, and thus do not present a soft surface to the skin. In addition, they prevent natural air flow to the skin.

There is a need for a disposable personal product with an effective absorbency level, which is also environmentally friendly, and which does not typically cause an adverse reaction on contact with the skin of a user.

Thus it is an object of the present invention to overcome, or at least partially alleviate, the difficulties associated with the prior art.

The word “disposable” is used in the specification to describe absorbent articles which are not intended to be laundered or otherwise restored or reused as an absorbent article. They are intended to be discarded after a single use and preferably to be disposed of in an environmentally friendly manner.

The phrase “personal product” is intended to include products or articles used to absorb waste body fluids or other body waste, and includes diapers, nappies, absorbent underpants, training pants, adult incontinence products, feminine hygiene products and breast pads. This phrase is also intended to cover products used as protectors for beds, chairs and baby cots which may come into contact with waste body discharges.

References to prior art in this specification are provided for illustrative purposes only and are not to be taken as an admission that such prior art is part of the common general knowledge in Australia or elsewhere.

### SUMMARY OF THE INVENTION

The present invention provides a non-woven disposable personal product comprising an absorbent layer of viscose.

The viscose is preferably untreated viscose such as unbleached viscose.

Unbleached viscose has the benefit of not containing any of the harsh chemicals typically used in the bleaching process. Therefore the use of unbleached viscose reduces the incidence of nappy rash which is often caused by harsh chemicals affecting the skin of the infant. In addition, the use of unbleached viscose allows for increased air circulation around the genital and bottom areas of the infant which is often a problem in the case of products containing synthetic materials and strong chemicals. Furthermore, the absence of strong chemicals in the unbleached viscose in the product allows for the easier and more natural absorption of urine or other waste body fluids in the product.

Alternatively, the viscose may be bleached viscose depending on the specific requirements of the product.

Preferably the viscose is in the form of viscose fluff. The viscose may be compressed viscose.

The non-woven disposable personal product preferably further comprises a layer of a biodegradable aliphatic polyester. The biodegradable aliphatic polyester is preferably polylactic acid (PLA).

The absorbent layer preferably further comprises a starch/polymer mixture to form an absorbent core. The viscose may be coated with the starch/polymer mixture to form the absorbent core.

In a preferred alternative, the starch/polymer is in the form of beads, grains or flakes dispersed within the viscose. The polymer starch beads are preferably small, granular particles around the size of powdered sugar grains or flakes. The relative weight percentage of the viscose:starch/polymer mixture in the absorbent core is preferably about 90%:7%.

The absorbent core may further comprise a small amount of a hydrogel mixed with the polymer/starch mixture to assist in maintaining the shelf life of the product by absorbing moisture when the product is on the shelf. Preferably the hydrogel is present in the absorbent core in an amount of less than about 3% by weight of the absorbent core in the case of baby and infant diapers. Preferably the hydrogel is present in an amount of about 7 to 20% in the absorbent core in the case of adult diapers and incontinence products.

More preferably the absorbent core has a composition with relative weight percentages of viscose fluff:starch/polymer:hydrogel of about 90% 7% 3%.

The use of hydrogel in absorbent products such as diapers allows the absorption of moisture (dampness) from the absorbent core. Since the personal product of the invention is made up of natural materials it is important to keep the absorbent materials in the product dry until they are required for use and ensure a good shelf life.

The absorbent core may comprise a first layer and a second layer, the second layer having a greater relative weight percentage of starch/polymer:viscose than the first layer. In use, the second layer is typically positioned away from the user so that liquid waste matter is partly absorbed by the first layer before reaching the second layer.

The absorbent core is preferably substantially rectangular in shape to conveniently collect urine and blood or other waste matter from the body of the user as excreted.

The starch used in the absorbent core is preferably formed by grafting a polymeric composition to corn starch.

The absorbent core may be surrounded by a layer of tissue paper.

The product may further comprise an outer layer in the form of an outer cover.

The outer cover serves to form a covering for retaining the absorbent core and other components of the product. The outer cover is preferably formed of any one or more of non-woven cotton, viscose, silk, bamboo or wool. The outer cover is preferably in the form of a non-woven felted fabric, such as a felted viscose fabric, and may be unbleached, bleached or partially bleached. The felted viscose fabric may be needle felted and may be produced by any one of the methods used in non-woven fabrics including random laid, parallel laid or cross laid. The use of the non-woven felted viscose fabric provides a soft, smooth feel to the skin.

The non-woven disposable personal product may further comprise a hydrophobic layer adjacent skin of the user to assist in repelling liquid away from the skin of the user. The product may optionally include a hydrophilic layer beneath the hydrophobic layer to assist in preventing the waste liquid absorbed by the absorbent core from being drawn back towards the skin. The product may also comprise a biodegradable sheet film to assist in waterproofing and retaining liquid matter within the product. The biodegradable film is typically positioned adjacent the absorbent core to assist in preventing the moisture from seeping through the outer cover. The biodegradable film preferably comprises polylactic acid (PLA).

In a further preferred form of the invention, the invention provides a non-woven disposable personal product comprising an absorbent core, the absorbent core comprising viscose and a starch/polymer mixture. The starch in the starch/polymer mixture is preferably in the form of starch grains or flakes, the grains or flakes being dispersed in the viscose.

Still further, the invention provides a non-woven disposable personal product comprising an outer cover, a hydrophobic layer adjacent skin of a user, and an absorbent core intermediate the outer cover and the hydrophobic layer, the absorbent core comprising viscose, a starch/polymer mixture and hydrogel. The starch in the starch/polymer mixture is preferably in the form of starch grains or flakes dispersed in the viscose. The product may further comprise a hydrophilic layer positioned beneath the hydrophobic layer. The hydrophilic layer and the hydrophobic layer preferably both comprise polylactic acid (PLA).

The invention further provides a composition for a non-woven disposable personal product comprising an absorbent layer of viscose and a layer of polylactic acid (PLA). Preferably the viscose is unbleached viscose.

Throughout the specification, unless the context requires otherwise, the word “comprise” or variations such as “comprises” or “comprising”, will be understood to imply the inclusion of a stated integer or group of integers but not the exclusion of any other integer or group of integers. Likewise the word “preferably” or variations such as “preferred”, will be understood to imply that a stated integer or group of integers is desirable but not essential to the working of the invention.

### BRIEF DESCRIPTION OF THE DRAWINGS

The present invention will now be described, by way of example only, with reference to the accompanying drawings, in which:

FIG. 1 is a schematic plan view of a diaper according to an embodiment of the invention; and

FIG. 2 is a schematic cross-sectional view through the diaper of FIG. 1.

### DESCRIPTION OF AN EMBODIMENT OF THE PRESENT INVENTION

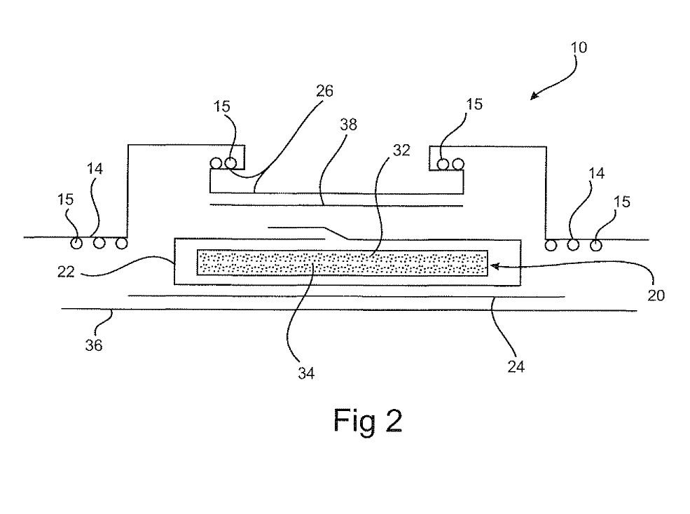

A diaper 10 for an infant having a pair of shaped leg portions 12 and elasticised cuff portions 14 is shown in the Figures. As can best be seen in FIG. 2, the cuff portions 14 have three rows of elastic 15 running the length of the diaper 10 so that the edges of the cuff portions 14 sit securely on the legs of the infant. Two further rows of elastic 15 may be provided on an extension of the cuff section 14 of the diaper 10 to assist in holding the diaper in position on the legs as shown in FIG. 2.

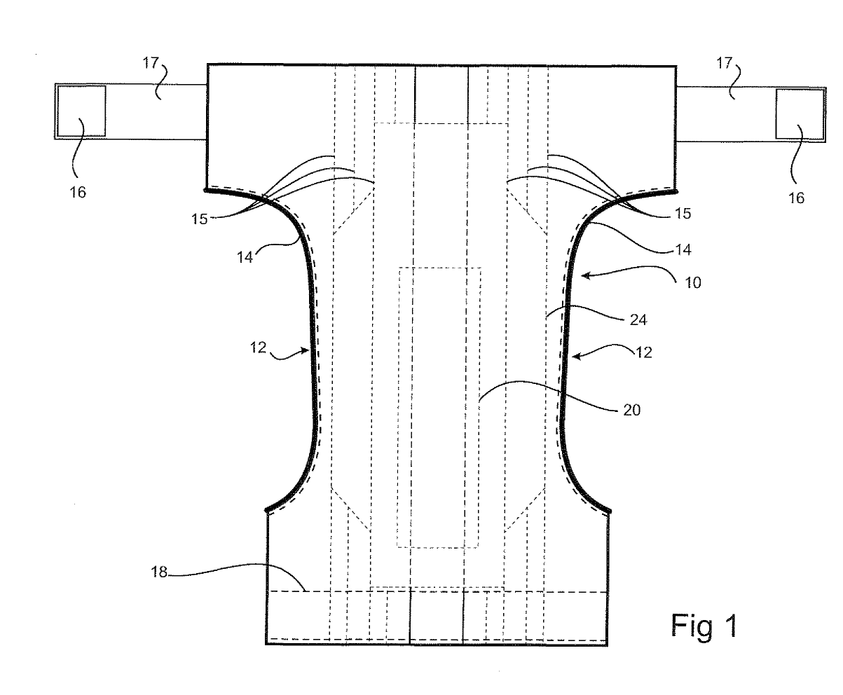

The diaper has two tape portions 17 fixed to the upper end of the diaper 10 as shown in FIG. 1. The tape portions 17 are provided with adhesive tab portions 16 for fixing to the tape region 18 to secure the diaper during use.

The diaper 10 has an absorbent core 20 comprised of starch grains/flakes 32 in a starch/polymer mixture dispersed in viscose fluff 34 as shown in FIG. 2. The absorbent core also includes hydrogel (not shown in the drawings). The relative weight percentage of viscose fluff:starch/polymer:hydrogel in the absorbent core 20 is about 90%:7%:3%. The presence of the hydrogel maintains the dryness of the product during its shelf life. Hydrogel is needed for this purpose due to the fact that the majority of the raw materials being used in the diaper are natural fibres/polymers which readily absorb moisture from the air whilst the product is on the shelf.

The usual level of superabsorbent polymer (SAP) used for moisture absorption in the absorbent core of prior art diapers is about 40%. Such high amounts of SAP have caused a number of health and environmental problems. However, in the case of infant products of the present invention the content of hydrogel is usually around 3%. The quantity of hydrogel used in the present invention at this amount is at a sufficient level to assist in absorption of moisture during shelf life.

In the case of diapers used for babies of infants, the typical percentage of hydrogel present is less than or equal to about 3%. In the case of adult diapers, incontinence pads, or sanitary products, the absorbent core preferably comprises around 7 to 20% of hydrogel due to the larger size of the product and the volume of moisture which needs to be absorbed.

The presence of polymer/starch grains/flakes dispersed in the viscose fluff provides an effective means for the absorption of fluids in the product. It is thought that when the water or waste fluid falls on the starch grains, polar repulsion between the molecules occurs which results in swelling of the particles and absorption of the water or fluid.

As an alternative, the absorbent core may comprise a first layer and a second layer, the first layer being closest to the body of the user having a lower relative weight percentage of starch/polymer:viscose than the second layer. In use, this allows some of the moisture to be retained in the first layer and the bulk of the moisture to be retained in the lower second layer, thereby keeping the bulk of the “wetness” away from the body of the user.

Starch in the form of beads/flakes provides an increased surface area for contact with moisture and assists in increasing the absorbency capacity of the diaper. The effect of incorporating the starch beads/flakes 32 in the viscose fluff 34 is to create a material that acts like a sponge capable of high absorption rates. The starch is capable of sustaining multiple “swell-shrink” cycles.

Viscose is a regenerated fibre. Cellulose fibre in its regenerated form is used to produce viscose. Viscose usually contains about 10 percent moisture when “dry” and may absorb up to about 21 percent moisture before feeling wet. Thus it provides a convenient substance for use in diapers and other like products that have a need for the absorption of fluids.

The main absorbent ingredient in the absorbent core is the starch in the form of starch grains/flakes. The starch used in the absorbent core is preferably formed by grafting a polymeric composition to corn starch molecules. Due to the nature of the starch, it is capable of absorbing up to 300 times its weight in water. The additional starch in the form of grains is substantially bio degradable and compostable.

The absorbent core 20 is surrounded by a layer of tissue paper 22.

The diaper 10 is provided with an outer cover 36 formed of non-woven felted viscose although other materials may also be suitable. The outer cover forms a covering for retaining the absorbent core and other components of the diaper. It is the layer which is furthermost away from the body of the user. The outer cover is soft and has a comfortable feel against the skin of a user. The nature of this fabric allows for breathability and ease of air flow around the skin of a user. The feel of the outer cover 36 is very smooth to the body of the user and provides ease of movement and natural air flow to the body. The natural fibre construction of the outer cover offers maximum health and environmental benefits.

The diaper 10 also has a hydrophobic layer 26 of non-woven material and an inner hydrophilic layer 38. The hydrophobic layer 26 provides for a substantially dry area next to the skin of the user. The hydrophobic layer 26 is typically a bonded, non-woven fabric which also forms the cuff. The hydrophobic layer 26 and the hydrophilic layer 38 are both comprised of a biodegradable thermoplastic, aliphatic polyester in the form of polylactic acid (PLA). To produce a suitable product for use in the invention, PLA methodology is used which includes the processing and treatment of the PLA to form a fibre of film-like material.

Polylactic acid (PLA) is particularly attractive as a sustainable alternative to petrochemical-derived products. The reason for this is that the lactate from which the PLA is ultimately produced can be derived from the fermentation of agricultural by-products such as corn starch, or other starch-rich products like maize, sugar cane, or wheat. Although PLA can be more expensive than many petroleum-derived commodity plastics, it has many benefits in the present invention. For example PLA breaks down faster than many other materials and this is the reason this material is used in medical applications such as for sutures or coatings in medicine capsules.

The elastic 15 is also of a hydrophobic nature and may be formed of the same bonded, non-woven fabric as the hydrophobic layer. This further assists in retaining dryness next to the skin of the user.

The diaper 10 is also conveniently provided with a biodegradable sheet film 24 which assists in waterproofing and retaining the absorbed fluid within the diaper 10. The biodegradable film 24 is typically positioned adjacent the absorbent core 20 as can be seen in FIG. 2, to assist in preventing the moisture from seeping through the outer cover 36. The film 24 is derived from lactic acid and is a breathable and waterproof film layer and therefore is beneficial to the overall design of the diaper 10. Alternatively, the film 24 of the present invention uses man made biodegradable aliphatic polyesters such as PLA in the construction of the film 24.

Man-made biodegradable aliphatic polyesters are based mainly on the industrial polymerisation of monomers such as glycolic acid (PGA), lactic acid (PLA), butyric acid (PHB), and so on. The formula for PLA which is repeated in long polymer chains is as follows:

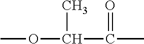

Unlike aromatic polyesters, the aliphatic polyesters are generally biodegradable. Biodegradation of aliphatic polyester fibres such as PLA occurs when their constituent polymers are depolymerised, usually by the action of enzymes secreted by micro-organisms. More than a hundred types of bacteria or micro-organisms are known to synthesise and store aliphatic polyesters such as PLA leading to the biodegradation of this material. The enzymes act by hydrolysing or oxidising the polymer, and can work on the ends of chains (exo-enzymes) or randomly along their length (endo-enzymes). To do its work, the enzyme has to be able to bond to the fibre and gain access to sites capable of being oxidised or hydrolysed. The most biodegradable fibres therefore tend to be hydrophilic and made up of short, flexible chains. They will often have chain back bones with oxygen (such as PLA) or nitrogen links and/or pendant groups containing oxygen or nitrogen atoms. Most fibres made of natural polymers fall into this group.

Now that preferred embodiments of the non-woven disposable personal product have been described in detail, it will be apparent that the embodiments provide a number of advantages over the prior art, including the following:

(i) They provide an effective personal product comprised of substantially natural or regenerated/reconstructed materials or fibres such that on disposal there is minimum adverse impact on the environment.
(ii) The use of non-woven materials in the product allows for ease of breakdown of the product in the environment.
(iii) The personal product is formed of natural materials and fibres to provide an effective and healthy product for the user.
(iv) The invention provides an environmentally friendly and generally allergy free composition and product due to the use of natural and regenerated materials.
(v) The soft outer cover of non-woven viscose presents a feeling of softness to the skin. The fabric allows ease of air circulation and breathability around the skin of the user.
(vi) The use of the biodegradable sheet film layer assists in retaining fluid in the diaper whilst at the same time conforming to the requirements of a natural environmentally friendly product.

It will be readily apparent to persons skilled in the relevant arts that various modifications and improvements may be made to the foregoing embodiments, in addition to those already described, without departing from the basic inventive concepts of the present invention. For example, the outer cover of the personal product may be comprised of materials other than viscose that also give a soft feel to the product, such as cotton, rayon, silk or wool, bamboo, milk fibre, or mixtures thereof. Therefore, it will be appreciated that the scope of the invention is not limited to the specific embodiments described.

1-37. (canceled)

38. A non-woven disposable personal product to be positioned adjacent skin of a user, the non-woven disposable personal product comprising:
an absorbent layer of viscose combined with a starch and polymer mixture to form an absorbent core;
a layer of a biodegradable aliphatic polyester including polylactic acid (PLA), and
a hydrophobic layer to be positioned adjacent the skin of the user to assist in drawing liquid away from the skin of the user.

39. A non-woven disposable personal product according to claim 38 wherein the viscose is unbleached viscose.

40. A non-woven disposable personal product according to claim 38 wherein the viscose is bleached viscose.

41. A non-woven disposable personal product according to claim 38 wherein the viscose is in the form of viscose fluff.

42. A non-woven disposable personal product according to claim 38 wherein the viscose is compressed viscose.

43. A non-woven disposable personal product according to claim 38 wherein the viscose is coated with the starch and polymer mixture to form the absorbent core.

44. A non-woven disposable personal product according to claim 43 wherein the starch and polymer mixture includes at least one of grains and flakes.

45. A non-woven disposable personal product according to claim 44 wherein the at least one of grains and flakes includes small, granular particles or flakes of starch.

46. A non-woven disposable personal product according to claim 44 wherein the at least one of grains and flakes are dispersed within the viscose.

47. A non-woven disposable personal product according to claim 38 wherein a relative weight percentage of viscose to the starch and polymer mixture in the absorbent core is 90 percent viscose and 7 percent starch and polymer mixture.

48. A non-woven disposable personal product according to claim 38 wherein the absorbent core further comprises hydrogel mixed with the starch.

49. A non-woven disposable personal product according to claim 48 wherein the hydrogel in comprises no more than 3 percent of the absorbent core.

50. A non-woven disposable personal product according to claim 48 wherein the hydrogel is present in the absorbent core in an amount in a range of 7 percent to 20 percent of the adsorbent core.

51. A non-woven disposable personal product according to claim 48 wherein the absorbent core has a composition with relative weight percentages of 90 percent being the viscose, 7 percent being the starch and polymer mixture and 3 percent being the hydrogel.

52. A non-woven disposable personal product according to claim 38 wherein the absorbent core comprises a first layer and a second layer, the second layer having a greater relative weight percentage of the starch and polymer mixture to the viscose than does the first layer.

53. A non-woven disposable personal product according to claim 38 wherein the absorbent core is rectangular.

54. A non-woven disposable personal product according to claim 38 wherein the starch and polymer mixture is formed by synthetically grafting a polymeric composition to corn starch.

55. A non-woven disposable personal product according to claim 38 wherein the absorbent core is surrounded by a layer of tissue paper.

56. A non-woven disposable personal product according claim 38 further comprising an outer layer in the form of an outer cover including at least one of cotton, viscose, silk, bamboo and wool of non-woven construction.

57. A non-woven disposable personal product according to claim 56 wherein the outer cover is formed of viscose and the viscose in the outer cover is manufactured in the form of a non-woven felted fabric.

58. A non-woven disposable personal product according to claim 57 wherein the non-woven felted fabric is a needle felted fabric.

59. A non-woven disposable personal product according to claim 38 wherein the hydrophobic layer comprises a biodegradable aliphatic polyester.

60. A non-woven disposable personal product according to claim 59, wherein the biodegradable aliphatic polyester is polylactic acid (PLA).

61. A non-woven disposable personal product according to claim 38 further comprising a hydrophilic layer beneath the hydrophobic layer.

62. A non-woven disposable personal product according to claim 61 wherein the hydrophilic layer comprises a biodegradable aliphatic polyester.

63. A non-woven disposable personal product according to claim 62, wherein the biodegradable aliphatic polyester is polylactic acid (PLA).

64. A non-woven disposable personal product according to claim 38 further comprising a biodegradable sheet film positioned adjacent the absorbent core, the biodegradable sheet film comprising polylactic acid (PLA).

65. A non-woven disposable personal product comprising:
an absorbent core comprising viscose and a mixture of a starch and a polymer;
a layer of a biodegradable aliphatic polyester, and
a hydrophobic layer to be positioned adjacent skin of a user.

66. A non-woven disposable personal product according to claim 65 wherein starch in the starch and polymer mixture is in the form of starch grains or starch flakes, the grains or flakes being dispersed within the viscose.
